(19) United States
(12) Patent Application Publication
(10) Pub. No.: US 2023/0173235 A1
(43) Pub. Date: Jun. 8, 2023

(54) METHOD AND APPARATUS FOR TREATMENT OF CHRONIC VENOUS INSUFFICIENCY

(71) Applicants: Boston Scientific Scimed, Inc., Maple Grove, MN (US); Regents of the University of Minnesota, Minneapolis, MN (US)

(72) Inventors: Ajay Gupta, Minneapolis, MN (US); Christian Angelopoulos, Minneapolis, MN (US); Sergei Grishankov, Minneapolis, MN (US)

(73) Assignees: Boston Scientific Scimed, Inc., Maple Grove, MN (US); Regents of the University of Minnesota, Minneapolis, MN (US)

(21) Appl. No.: 18/073,263

(22) Filed: Dec. 1, 2022

Related U.S. Application Data
(60) Provisional application No. 63/285,552, filed on Dec. 3, 2021.

(51) Int. Cl. A61M 25/10; A61F 2/24; A61L 29/02; C08L 83/04

(52) U.S. Cl. CPC A61M 25/10184; A61F 2/2475; A61L 29/02; C08L 83/04; A61M 2025/1043

(57) ABSTRACT

A catheter may be used for treating chronic venous insufficiency (CVI) by remodeling a venous valve within a vein. The catheter is advanced through the vein to a position proximate the venous valve, the catheter including a hollow needle extendable out a first side wall of the catheter and an inflatable balloon located on a second, opposing, side wall of the catheter. The inflatable balloon is inflated in order to urge the catheter towards a vessel wall opposite that of the inflatable balloon. The hollow needle is advanced at least partially into the vessel wall opposite that of the inflatable balloon and a bulking agent is injected near the venous valve in order to remodel the venous valve. The bulking agent may be injected within a subintimal space within the vessel wall, or into an advential space outside of the vessel wall.

### CROSS-REFERENCE TO RELATED APPLICATIONS

This application claims the benefit of priority under 35 U.S.C. §119 of U.S. Provisional Application No 63/285,552, filed Dec. 3, 2021, the entire disclosure of which is hereby incorporated by reference.

### TECHNICAL FIELD

The present disclosure pertains to medical devices, and methods for manufacturing and using medical devices. More particularly, the disclosure is directed to treatment of chronic venous insufficiency (CVI).

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### SUMMARY

This disclosure provides design, material, manufacturing method, and use alternatives for medical devices. As an example, a catheter is adapted for remodeling a venous structure within a patient’s leg. The catheter includes an elongate shaft extending to a distal region, the distal region including a first side wall and an opposing second side wall. A needle lumen extends through the elongate shaft to a needle lumen aperture disposed within the first side wall and a hollow needle is slidingly disposed within the needle lumen such that the hollow needle is able to extend through the needle lumen aperture, the hollow needle adapted to be operably coupled with a source of remodeling material. An inflatable balloon is secured to the second side wall of the elongate shaft, and an inflation lumen extends through the elongate shaft and fluidly coupled with the inflatable balloon, the inflation lumen adapted to be operably coupled with a source of inflation fluid.

Alternatively or additionally, the inflatable balloon may be adapted such that inflating the inflatable balloon causes the inflatable balloon to engage a vessel wall and push the distal region of the elongate shaft away from that vessel wall.

Alternatively or additionally, the hollow needle may be adapted to extend only partially into a vessel wall when remodeling a venous structure including a venous valve.

Alternatively or additionally, the hollow needle may be adapted to extend entirely through a vessel wall when remodeling a venous structure including a vein.

Alternatively or additionally, the venous structure may include a vein just below a venous valve.

Alternatively or additionally, the remodeling material may include medical grade silicone.

As another example, a method of treating chronic venous insufficiency remodels a venous valve disposed within a vein within a patient’s leg. The method includes advancing a catheter through the vein to a position proximate the venous valve, the catheter including a hollow needle extendable out a first side wall of the catheter and an inflatable balloon located on a second, opposing, side wall of the catheter. The inflatable balloon is inflated in order to urge the catheter towards a vessel wall opposite that of the inflatable balloon. The hollow needle is advanced at least partially into the vessel wall opposite that of the inflatable balloon and a bulking agent is injected near the venous valve in order to remodel the venous valve.

Alternatively or additionally, advancing the hollow needle at least partially into the vessel wall may include advancing the hollow needle through an endothelium of the vessel wall and into a subintimal space within the vessel wall.

Alternatively or additionally, injecting the bulking agent near the venous valve may include injecting the bulking agent into the subintimal space within the vessel wall.

Alternatively or additionally, advancing the hollow needle at least partially into the vessel wall may include advancing the hollow needle through the vessel wall and into an advential space exterior to the vessel wall.

Alternatively or additionally, injecting the bulking agent near the venous valve may include injecting the bulking agent into the advential space exterior to the vessel wall.

Alternatively or additionally, the method may further include using external ultrasound to evaluate progress towards remodeling the venous valve.

Alternatively or additionally, the method may further include withdrawing the hollow needle back into the catheter, moving the catheter to a different position, advancing the hollow needle at least partially into the vessel wall, and injecting a bulking agent near the venous valve.

Alternatively or additionally, moving the catheter to a different position may include deflating the inflatable balloon prior to moving the catheter to a different position, moving the catheter to a different position, and inflating the inflatable balloon prior to advancing the hollow needle at least partially into the vessel wall.

Alternatively or additionally, moving the catheter to a different position may include translating the catheter and/or rotating the catheter.

As another example, a method of treating chronic venous insufficiency remodels a venous valve disposed within a vein within a patient’s leg, the method. The method includes advancing a catheter through the vein to a position proximate the venous valve, the catheter including a hollow needle extendable out a first side wall of the catheter and an inflatable balloon located on a second, opposing, side wall of the catheter. The inflatable balloon is inflated in order to urge the catheter towards a vessel wall opposite that of the inflatable balloon. The hollow needle is advanced into the vessel wall opposite that of the inflatable balloon, the hollow needle advancing through the vessel wall and into an advential space outside the vessel wall. A bulking agent is injected into the advential space within the vessel wall.

Alternatively or additionally, advancing the catheter through the vein may include advancing the catheter through the vein from a position below the venous valve.

Alternatively or additionally, the method may further include withdrawing the hollow needle back into the catheter, moving the catheter to a different position, advancing the hollow needle into and through the vessel wall, and injecting a bulking agent into the advential space outside the vessel wall.

Alternatively or additionally, moving the catheter to a different position may include deflating the inflatable balloon prior to moving the catheter to a different position, moving the catheter to a different position, and inflating the inflatable balloon prior to advancing the hollow needle into and through the vessel wall.

Alternatively or additionally, moving the catheter to a different position may include translating the catheter and/or rotating the catheter.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic view of an illustrative venous valve undergoing remodeling;

FIG. 2 is a schematic view of an illustrative venous valve undergoing remodeling;

FIG. 3 is a schematic view of an illustrative catheter useable for venous valve remodeling;

FIG. 4 is a schematic view showing a venous valve before and after remodeling;

FIG. 5 is a flow diagram showing an illustrative method of remodeling a venous valve;

FIG. 6 is a flow diagram showing an illustrative method of remodeling a venous valve;

FIG. 7 is a side view of an illustrative catheter useable for venous valve remodeling; and

FIG. 8 is a side view of an illustrative catheter useable for venous valve remodeling.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Chronic venous insufficiency (CVI) is a condition in which poorly functioning venous valves, such as in a patient’s legs, can lead to a variety of problems. For example, CVI can lead to problems such as lower extremity lymphedema, varicose veins, DVT (deep vein thrombosis), edema, pain and ulcers. During early stages of chronic venous insufficiency, there is mild valvular dysfunction, which progressively worsens if left untreated. Valve dysfunction can be a large driver of thrombus formation, which can permanently destroy the valve and lead to progressively worse stages of chronic venous insufficiency. The extra fluid buildup in the patient’s legs can overload the lymphatic system, causing progressive deterioration and leading to lymphedema (sometimes called phlebolymphedema).

At relatively early stages, it is estimated that 30 to 50 percent of venous valves retain functioning valve leaflets, but venous wall weakness and local dilation can prevent the valve leaflets from closing properly. Usually a proximal valve (closer to the abdomen) becomes incompetent first, which subjects the venous valves below it to greater pressures. In time, these increased pressures cause the distal valves to also fail.

While any venous valves, including the valves in the deep and superficial venous systems as well as perforators can be treated using the techniques described herein, the proximal valves such as the Femoral valves, valves at the Great Saphenous valve/Common femoral valve junction and popliteal valves are well suited for the techniques described herein.

About half the time, deep venous reflux is the root problem, but only superficial venous insufficiency is currently treated by stripping or sclerosing the vein. Valves can be reconstructed successfully, but only with open surgery, which is generally not performed at earlier stages of the disease. Deep venous reflux can be detected and quantified using external duplex ultrasound. Using techniques such as Valsalva maneuver, distal augmentation, abdominal pressure and others, a skilled sonographer can detect early stages of reflux disease, and discount other, non-venous wall elated etiologies in an office setting.

Veins are easily deformable unless calcified or unless they contain a thrombus. Native venous valves are not round, but rather are elliptical. Restoring their shape can reduce reflux. By observing ultrasound waveforms and manipulation techniques, a skilled sonologist can diagnose patients having primary disease, and not proximal occlusion (continuous waveform, less responsive to respiration), left heart fluid overload (pulsatile flow corresponding to heartbeat) or DVT (deep vein thrombosis, sonographic evidence and symptoms). In some cases, the pressure in the vein caudal to the valve can also be measured.

Leaflet function and orientation can be viewed using transverse-coronal views. The major axis of the elliptical valve (and the direction of the leaflets) is usually parallel to the fascia and skin. The physician can gain access to the popliteal vein, distal tibial, lesser saphenous or other lower leg veins using ultrasound that leads to the vein with the target valve. This can be done with needle, wires, insertion sheath and the like under ultrasound guidance. The patient can be positioned to optimize access, operator comfort and physiological need. The access point can be determined based on the size of the insertion sheath needed and anatomical features such as the tortuosity of the vasculature.

The valve in question can be reshaped by applying external pressures. For example, the leg can be compressed using a clamp until reflux improves, or the valve can be stretched internally along the major axis. In some cases, a variety of different external support structures can be used to help reshape the valve, including but not limited to springs, clamps, wraps, compressive threads, bulking material, selective ablation, rods, patches and the like.

A bulking agent may be injected either within the subintimal space, or outside of the venous wall in the advential space. If the bulking agent is injected within the subintimal space, the greater relative strength of the outer layers of the vessel wall means that the bulking agent will force the endothelium inwards towards the lumen. Alternatively, in some cases, chemicals or laser light may be used to cause connective tissue to denature, fibrose or harden.

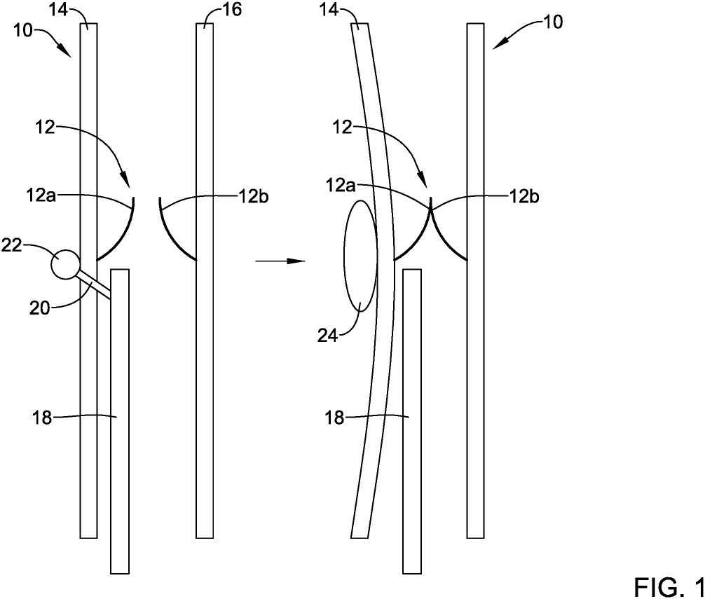

FIG. 1 is a schematic view of an illustrative venous valve undergoing remodeling. On the left side of FIG. 1 is a vein 10 having a venous valve 12 disposed within the vein 10. The venous valve 12 includes a first leaflet 12a and a second leaflet 12b. As can be seen, the first leaflet 12a and the second leaflet 12b do not contact each other, even when in a “closed” position. This allows blood to leak back through the venous valve 12, which is known as reflux.

The vein 10 can be seen as having a first vessel wall 14 and a second vessel wall 16. In looking at the vein 10 on the left side of FIG. 1, it can be seen that the distance between the first vessel wall 14 and the second vessel wall 16 is greater than can be successfully spanned by the first leaflet 12a and the second leaflet 12b of the venous valve 12. In comparison, by looking at the vein 10 as shown on the right side of FIG. 1, it can be seen that the first vessel wall 14 has been pushed towards the second vessel wall 16, thereby reducing the relative distance between the first vessel wall 14 and the second vessel wall 16. As a result, the first leaflet 12a and the second leaflet 12b of the venous valve 12 can now successfully span the distance therebetween. Consequently, the venous valve 12 can now function appropriately, doing a better job of preventing blood flow when the venous valve 12 is closed, thereby reducing or even eliminating reflux.

On the left side of FIG. 1, a catheter 18 is positioned close to the first vessel wall 14. While not shown in this drawing, the catheter 18 may include structure that can be used to bias the catheter 18 into proximity with (as shown) the first vessel wall 14. The catheter 18 includes a hollow needle 20 that extends laterally from a side of the catheter 18. The hollow needle 20 may be considered as being in fluid communication with a source of a bulking agent, as a small area 22 of bulking agent has been deposited outside of the vein 10, adjacent to the first vessel wall 14.

In looking at the right side of FIG. 1, it can be seen that while the catheter 18 has not yet been withdrawn, the hollow needle 20 has been withdrawn back into the catheter 18 in preparation for removal of the catheter 18. Now there is a more substantial area 24 of bulking agent that has been deposited outside of the vein 10, adjacent to the first vessel wall 14. As can be seen, the more substantial area 24 of bulking agent has pressed the first vessel wall 14 towards the second vessel wall 16, thereby reducing the distance between the first vessel wall 14 and the second vessel wall 16. As shown, the more substantial area 24 of bulking agent is entirely outside of the first vessel wall 14. In some cases, the bulking agent may instead be deposited within the subintimal space (inside the endothelium) of the first vessel wall 14.

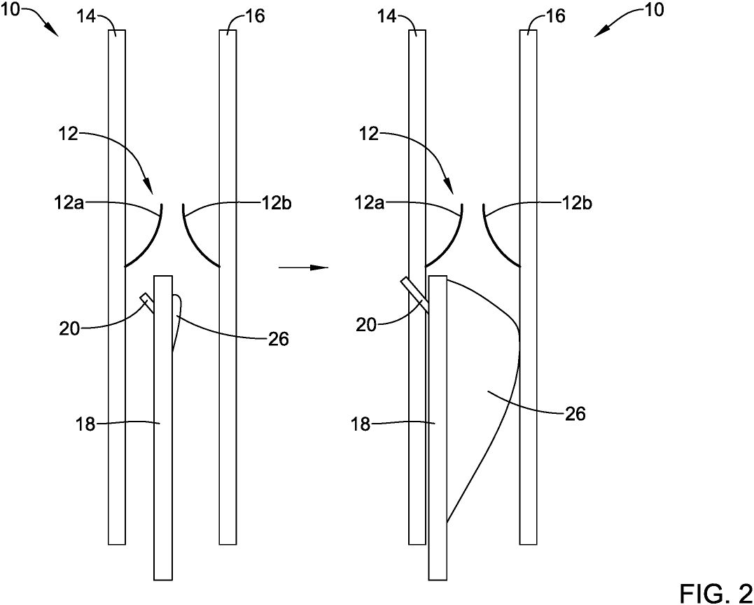

FIG. 2 may be considered as another view of the “before”, or the left side of FIG. 1. As can be seen, the catheter 18 includes an inflatable balloon 26 that is on an opposite side of the catheter 18 relative to where the hollow needle 20 is. Accordingly, and as seen on the right side of FIG. 2, the inflatable balloon 26 has been inflated. The inflatable balloon 26 engages with the second vessel wall 16 and pushes the catheter 18 closer to the first vessel wall 14 so that the hollow needle 20 is able to penetrate into the first vessel wall 14. In this, it will be appreciated that first and second are arbitrary, and the catheter 18 could be reversed, with the inflatable balloon 26 engaging the first vessel wall 14 and urging the catheter 18, and hence the hollow needle 20, into contact with the second vessel wall 16.

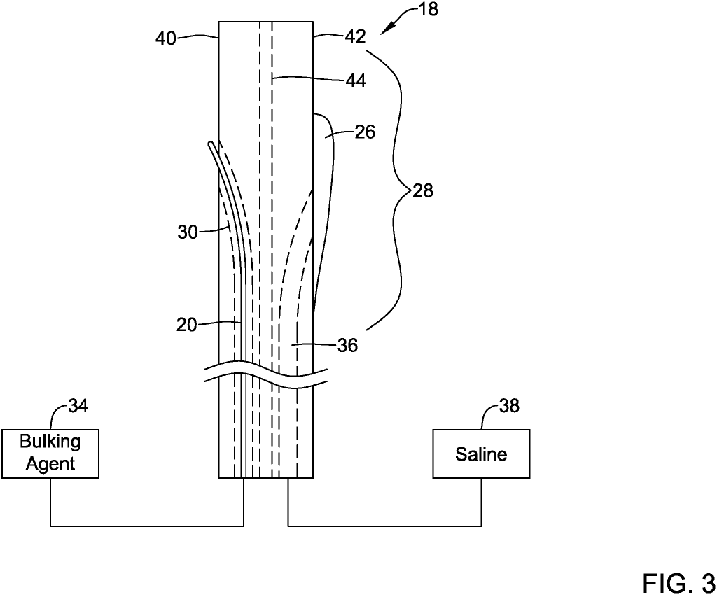

FIG. 3 is a schematic view of the catheter 18, showing additional features. The catheter 18 may be considered as including a distal region 28. The catheter 18 includes a needle lumen 30 that extends through the distal region 28 and terminates at a needle lumen aperture 32. The hollow needle 20 is able to extend out of the needle lumen 30 through the needle lumen aperture 32. The hollow needle is in fluid communication with a source 34 of bulking agent. While not pictured, it will be appreciated that there may be one or more controllable valves that regulate flow of the bulking agent from the source 34 to the hollow needle 20. In some cases, the bulking agent may be medical grade silicone. The bulking agent may be cyano-acrylate adhesive, for example.

Examples of suitable types of bulking agents include liquids such as viscous liquids, gels, foams, fluidized biomaterials, and other materials. In some cases, viscous liquids having an injection viscosity of between about 1 and about 1,000 ,000 cP in a cannula ranging between about 18 to about 35 gauge may be used as a bulking agent. In some cases, viscous liquids having an injection viscosity of between about 50,000 and about 200,000 cP in a cannula ranging between about 23 to about 27 gauge may be used as a bulking agent.

In some cases, bulking agent may include gellan gum, which is a water-soluble anionic polysaccharide. The bulking agent may include non-animal stabilized hyaluronic acid, and may be cross-linked or not cross-linked. In some cases, the bulking agent may include cross-linked PEG (polyethylene glycol) hydrogels that are formed by cross-linking the gel in vitro, followed by grinding into a paste to make it injectable. In some cases, the bulking agent may be poly dimethyl siloxane. The bulking agent may be calcium hydroxylapatite, such as Coaptite™, which is a urethral bulking product available commercially from Boston Scientific. In some cases, the bulking agent may be considered as being a persistent bulking agent.

In some cases, carboxymethyl cellulose hydrogel and alginate may be used as a bulking agent. In some cases, a hydrogel containing between about 4% alginate and about 30% alginate may be used as a bulking agent. For example, a hydrogel containing about 17.5% alginate may be used as a bulking agent. In some cases, a crosslinking agent can also be included with the bulking agent in order to facilitate water retention and increase the time during which the bulking agent remains in the target site. If included in the bulking agent, any suitable electrolyte can be used. For example, a salt, such as MgCl₂, may be included at a concentration of between about 1 mol/L and about 6 mol/L. Other salts that may be used include MgCl₂, CaCl₂, SrCh, BaCh, Al₃SO₄, BaSO₄, CaCO₃, Ferric chloride, and Ferrous chloride. Additional details regarding such bulking agents may be found in US 10,512,751 and US 10,173,027, which references are incorporated by reference herein. Additional bulking agents are described in US 9,987,221; US 9,526,804; and US 9,017,361, which references are incorporated by reference herein.

An inflation lumen 36 extends through the distal region 28 and is in fluid communication with an interior of the inflatable balloon 26 such that an inflation fluid such as saline can be pumped into the interior of the inflatable balloon 26 in order to inflate the inflatable balloon 26, or removed from the interior of the inflatable balloon 26 in order to deflate the inflatable balloon 26. The inflation lumen 36 is fluidly coupled with a source 38 of saline for inflating and deflating the inflatable balloon 26. While not pictured, it will be appreciated that there may be one or more controllable valves that regulate flow of saline from the source 38 to the inflatable balloon 26, and vice versa.

The distal region 28 of the catheter 18 includes a first side wall 40 and a second side wall 42. As can be seen, the needle lumen aperture 32 is disposed on the first side wall 40. The inflatable balloon 26 is disposed on the second side wall 42. By placing the inflatable balloon 26 on the second side wall 42, opposite the needle lumen aperture 32 on the first side wall 40, the relative position of the needle lumen aperture 32 relative to the first vessel wall 14, can be controlled by inflating and/or deflating the inflatable balloon 26. In some cases, the catheter 18 may include a guidewire lumen 44 that enables the catheter 18 to be advanced over a guidewire (not shown).

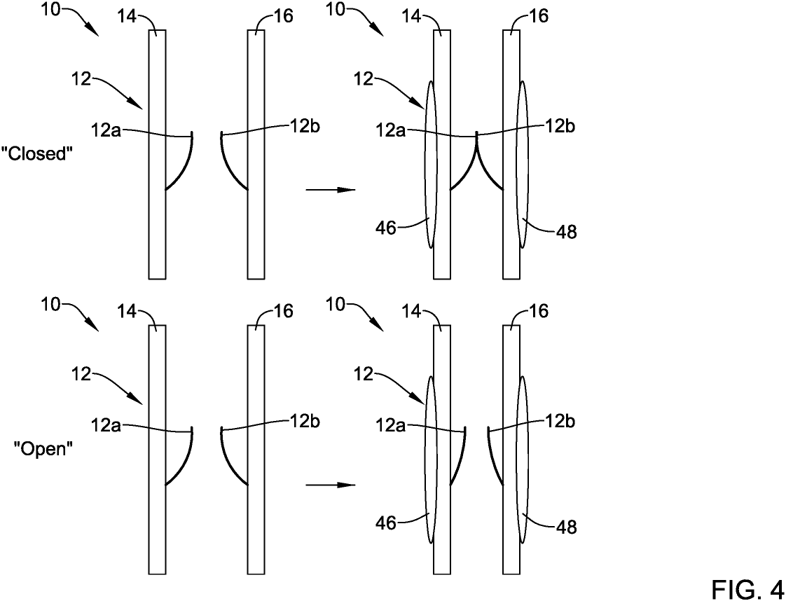

FIG. 4 is a schematic view showing how a venous valve has been improved by remodeling. In FIG. 4, the “closed” position of the venous valve 12 before remodeling is seen in the upper left of FIG. 4, where the distance between the first vessel wall 14 and the second vessel wall 16 is greater than can be successfully spanned by the first leaflet 12a and the second leaflet 12b. The lower left of FIG. 4 shows the “open” position of the venous valve 12 before remodeling.

The right side of FIG. 4 shows what has happened as a result of remodeling. The upper right of FIG. 4 shows the “closed” position of the venous valve 12 after remodeling. As can be seen, the relative distance between the first vessel wall 14 and the second vessel wall 16 has been reduced to the point that the first leaflet 12a and the second leaflet 12b of the venous valve 12 can span the distance, thereby reducing or even eliminating reflux. The lower right of FIG. 4 shows the “open” position of the venous valve 12 after remodeling.

In FIG. 4, multiple areas of bulking agent can be seen. As shown, there is a first area 46 of bulking agent disposed outside of the first vessel wall 14 and a second area 48 of bulking agent disposed outside of the second vessel wall 16. There may be two distinct areas of bulking agent, or the bulking agent may extend up to 360 degrees circumferentially around the vein 10, for example. It will be appreciated that this is a two dimensional picture of a three dimensional vein 10, after all. The catheter 18 can be positioned to inject bulking agent at a first location, and then the catheter 18 can be repositioned at a second location before injecting additional bulking agent, for example.

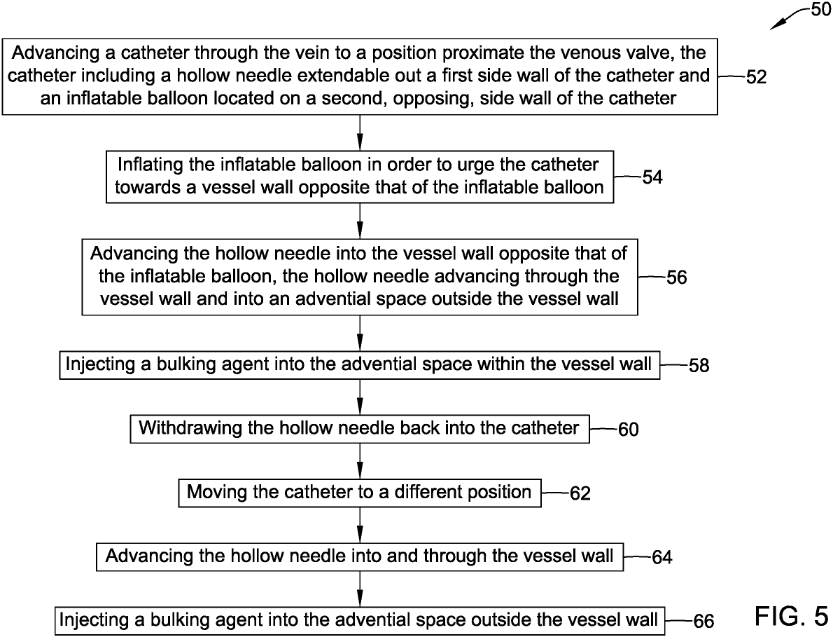

FIG. 5 is a flow diagram showing an illustrative method 50 of treating chronic venous insufficiency by remodeling a venous valve that is disposed within a vein within a patient’s leg. A catheter is advanced through the vein to a position proximate the venous valve, the catheter including a hollow needle extendable out a first side wall of the catheter and an inflatable balloon located on a second, opposing, side wall of the catheter, as indicated at block 52. The inflatable balloon is inflated in order to urge the catheter towards a vessel wall opposite that of the inflatable balloon, as indicated at block 54. The hollow needle is advanced at least partially into the vessel wall opposite that of the inflatable balloon, as indicated at block 56. A bulking agent is injected near the venous valve in order to remodel the venous valve, as indicated at block 58.

In some cases, advancing the hollow needle at least partially into the vessel wall may include advancing the hollow needle through an endothelium of the vessel wall and into a subintimal space within the vessel wall. Injecting the bulking agent near the venous valve may include injecting the bulking agent into the subintimal space within the vessel wall. In some cases, advancing the hollow needle at least partially into the vessel wall may include advancing the hollow needle through the vessel wall and into an advential space exterior to the vessel wall. Injecting the bulking agent near the venous valve may include injecting the bulking agent into the advential space exterior to the vessel wall.

In some instances, the hollow needle is withdrawn back into the catheter, as indicated at block 60. The catheter is moved to a different position, as indicated at block 62. The hollow needle is advanced at least partially into the vessel wall, as indicated at block 64. A bulking agent is injected, at the different position, near the venous valve, as indicated at block 66. In some cases, moving the catheter to a different position includes deflating the inflatable balloon prior to moving the catheter to a different position, moving the catheter to a different position, and inflating the inflatable balloon prior to advancing the hollow needle at least partially into the vessel wall. In some cases, moving the catheter to a different position may include translating the catheter and/or rotating the catheter. In some cases, external ultrasound may be used to evaluate progress towards remodeling the venous valve.

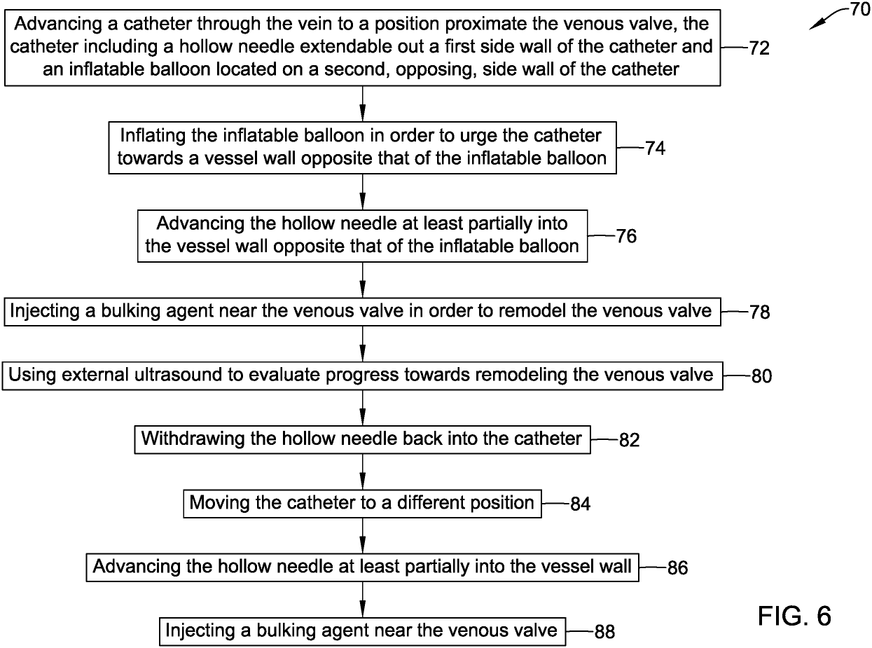

FIG. 6 is a flow diagram showing an illustrative method 70 of treating chronic venous insufficiency by remodeling a venous valve disposed within a vein within a patient’s leg. A catheter is advanced through the vein to a position proximate the venous valve, the catheter including a hollow needle extendable out a first side wall of the catheter and an inflatable balloon located on a second, opposing, side wall of the catheter, as indicated at block 72. In some cases, advancing the catheter through the vein may include advancing the catheter through the vein from a position below the venous valve.

The inflatable balloon is inflated in order to urge the catheter towards a vessel wall opposite that of the inflatable balloon, as indicated at block 74. The hollow needle is advanced into the vessel wall opposite that of the inflatable balloon, the hollow needle advancing through the vessel wall and into an advential space outside the vessel wall, as indicated at block 76. A bulking agent is injected into the advential space within the vessel wall, as indicated at block 78. In some cases, external ultrasound is used to evaluate progress towards remodeling the venous valve.

In some cases, the hollow needle is withdrawn back into the catheter, as indicated at block 82. The catheter is moved to a different position, as indicated at block 84. The hollow needle is advanced into and through the vessel wall, as indicated at block 86. A bulking agent is injected, at the different position, into the advential space outside the vessel wall, as indicated at block 88. In some cases, moving the catheter to a different position may include deflating the inflatable balloon prior to moving the catheter to a different position, moving the catheter to a different position, and inflating the inflatable balloon prior to advancing the hollow needle into and through the vessel wall. In some cases, moving the catheter to a different position may include translating the catheter and/or rotating the catheter.

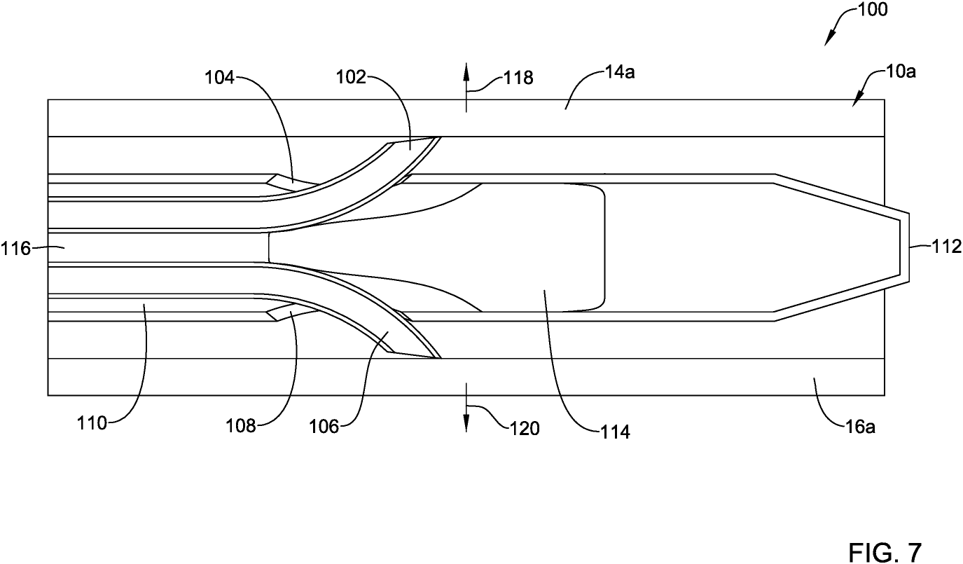

FIG. 7 is a side view of an illustrative catheter 100 that may be used in remodeling a venous valve. The illustrative catheter 100 is shown disposed within a vein 10a. The catheter 100 includes a first hollow needle 102 that extends out a first needle aperture 104 and a second hollow needle 106 that extends out a second needle aperture 108. The first hollow needle 102 and the second hollow needle 106 extend proximally through a central lumen 110 extending within the catheter 100. The catheter 100 terminates in an atraumatic tip 112.

The first hollow needle 102 and the second hollow needle 104 extend proximally to a position at which the first hollow needle 102 and/or the second hollow needle 104 can be caused to move distally or proximally, as desired. The catheter 100 includes a wedge 114 that is operably coupled with an elongate member 116. By pulling the elongate member 116 proximally, the wedge 114 engages the first hollow needle 102 and the second hollow needle 104 and urges the first hollow needle 102 in a first lateral direction indicated by an arrow 118 and urges the second hollow needle 104 in a second lateral direction indicated by an arrow 120. As a result, the first hollow needle 102 can penetrate into a first vessel wall 14a and the second hollow needle 104 can penetrate into a second vessel wall 16a. This means that bulking agent can be pumped into subintimal spaces of the vein 10a or even into the advential spaces outside the vein 10a at two different locations at the same time.

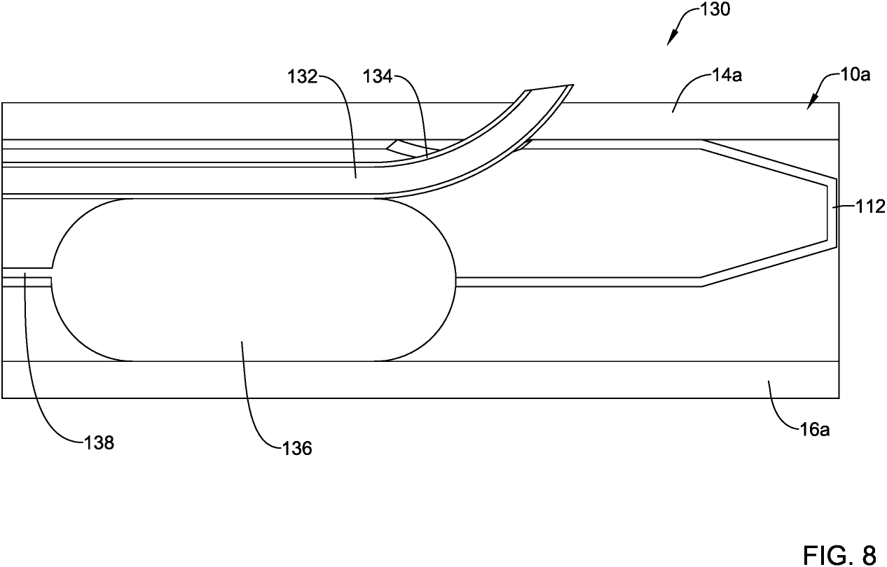

FIG. 8 is a side view of an illustrative catheter 130 that may be used in remodeling a venous valve. The illustrative catheter 130 is shown disposed within a vein 10a. The catheter 130 includes a hollow needle 132 that extends out a needle aperture 134 and into and even through the first vessel wall 14a. The catheter 130 includes an inflatable balloon 136 that is in fluid communication with an inflation lumen 138. By inflating the inflatable balloon 136, the catheter 130 is urged into contact with the first vessel wall 14a as a result of the inflatable balloon 136 engaging the second vessel wall 16a.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A catheter adapted for remodeling a venous structure within a patient’s leg, the catheter comprising:
an elongate shaft extending to a distal region, the distal region including a first side wall and an opposing second side wall;
a needle lumen extending through the elongate shaft to a needle lumen aperture disposed within the first side wall;
a hollow needle slidingly disposed within the needle lumen such that the hollow needle is able to extend through the needle lumen aperture, the hollow needle adapted to be operably coupled with a source of remodeling material;
an inflatable balloon secured to the second side wall of the elongate shaft; and
an inflation lumen extending through the elongate shaft and fluidly coupled with the inflatable balloon, the inflation lumen adapted to be operably coupled with a source of inflation fluid.

2. The catheter of claim 1, wherein the inflatable balloon is adapted such that inflating the inflatable balloon causes the inflatable balloon to engage a vessel wall and push the distal region of the elongate shaft away from that vessel wall.

3. The catheter of claim 1, wherein the hollow needle is adapted to extend only partially into a vessel wall when remodeling a venous structure comprising a venous valve.

4. The catheter of claim 1, wherein the hollow needle is adapted to extend entirely through a vessel wall when remodeling a venous structure comprising a vein.

5. The catheter of claim 4, wherein the venous structure comprises a vein just below a venous valve.

6. The catheter of claim 1, wherein the remodeling material comprises medical grade silicone.

7. A method of treating chronic venous insufficiency by remodeling a venous valve disposed within a vein within a patient’s leg, the method comprising:
advancing a catheter through the vein to a position proximate the venous valve, the catheter including a hollow needle extendable out a first side wall of the catheter and an inflatable balloon located on a second, opposing, side wall of the catheter;
inflating the inflatable balloon in order to urge the catheter towards a vessel wall opposite that of the inflatable balloon;
advancing the hollow needle at least partially into the vessel wall opposite that of the inflatable balloon; and
injecting a bulking agent near the venous valve in order to remodel the venous valve.

8. The method of claim 7, wherein advancing the hollow needle at least partially into the vessel wall comprises advancing the hollow needle through an endothelium of the vessel wall and into a subintimal space within the vessel wall.

9. The method of claim 8, wherein injecting the bulking agent near the venous valve comprises injecting the bulking agent into the subintimal space within the vessel wall.

10. The method of claim 7, wherein advancing the hollow needle at least partially into the vessel wall comprises advancing the hollow needle through the vessel wall and into an advential space exterior to the vessel wall.

11. The method of claim 10, wherein injecting the bulking agent near the venous valve comprises injecting the bulking agent into the advential space exterior to the vessel wall.

12. The method of claim 10, further comprising using external ultrasound to evaluate progress towards remodeling the venous valve.

13. The method of claim 10, further comprising:
withdrawing the hollow needle back into the catheter;
moving the catheter to a different position;
advancing the hollow needle at least partially into the vessel wall; and
injecting a bulking agent near the venous valve.

14. The method of claim 13, wherein moving the catheter to a different position comprises:
deflating the inflatable balloon prior to moving the catheter to a different position;
moving the catheter to a different position; and
inflating the inflatable balloon prior to advancing the hollow needle at least partially into the vessel wall.

15. The method of claim 14, wherein moving the catheter to a different position comprises translating the catheter and/or rotating the catheter.

16. A method of treating chronic venous insufficiency by remodeling a venous valve disposed within a vein within a patient’s leg, the method comprising:
advancing a catheter through the vein to a position proximate the venous valve, the catheter including a hollow needle extendable out a first side wall of the catheter and an inflatable balloon located on a second, opposing, side wall of the catheter;
inflating the inflatable balloon in order to urge the catheter towards a vessel wall opposite that of the inflatable balloon;
advancing the hollow needle into the vessel wall opposite that of the inflatable balloon, the hollow needle advancing through the vessel wall and into an advential space outside the vessel wall; and
injecting a bulking agent into the advential space within the vessel wall.

17. The method of claim 16, wherein advancing the catheter through the vein comprises advancing the catheter through the vein from a position below the venous valve.

18. The method of claim 16, further comprising:
withdrawing the hollow needle back into the catheter;
moving the catheter to a different position;
advancing the hollow needle into and through the vessel wall; and
injecting a bulking agent into the advential space outside the vessel wall.

19. The method of claim 18, wherein moving the catheter to a different position comprises:
deflating the inflatable balloon prior to moving the catheter to a different position;
moving the catheter to a different position; and
inflating the inflatable balloon prior to advancing the hollow needle into and through the vessel wall.

20. The method of claim 19, wherein moving the catheter to a different position comprises translating the catheter and/or rotating the catheter.
